(19) United States
(12) Patent Application Publication
(10) Pub. No.: US 2006/0060201 A1
(43) Pub. Date: Mar. 23, 2006

(54) Tracheostomy tube with improved tie and method holding same

(76) Inventors: Amy L. Beagle, Traverse City, MI (US); Robin E. Johnson, Glen Arbor, MI (US)

Correspondence Address: MILLER JOHNSON SNELL CUMMISKEY, PLC, 800 CALDER PLAZA BUILDING, 250 MONROE AVE N W, GRAND RAPIDS, MI, 49503-2250, US

(21) Appl. No.: 10/947,871

(22) Filed: Sep. 23, 2004

(51) Int. Cl. A62B 9/06; A61M 16/00

(52) U.S. Cl. 128/207.14; 128/207.17

(57) ABSTRACT

A tracheostomy tube tie apparatus 300 is used with a tracheostomy tube tie for attachment to a tracheostomy tube about a patient's neck. A cord lock fastener (301) is integrated with a portion of the tracheostomy tube flange (303) and attaches to the tracheostomy tube tie for slidably adjusting the diameter of the tracheostomy tube tie in order to hold the tracheostomy tube in a fixed position. The invention enables a single caregiver the ability to replace a tracheostomy tube about the patient's neck in an urgent situation using only one hand.

### TECHNICAL FIELD

This invention relates in general to a tracheostomy tube and more particularly to a tracheostomy tube whose tie is capable of being fastened by one caregiver.

### BACKGROUND

Tracheostomy tubes are well known in the art and are typically small cylindrical tubes which are designed to be placed directly into a patient's trachea through the front of the neck. A tracheostomy tube is often needed in situations where the patient has difficulty breathing and requires long-term mechanical ventilation, where the patient is unable to cough effectively and clear bodily secretions, or for patients with obstructed or blocked airways. If the tracheostomy tube is not changed on a periodic basis, infection and other medical problems can result.

As seen in prior art FIG. 1, a tracheostomy tube 100 typically includes a cannula 101 which is inserted through the stoma into the patient's trachea. The cannula typically is a long flexible material such as silicone or the like which acts to pass gas from an oxygen source (not shown) to the patient's lungs. The tracheostomy tube 100 further includes a fastener 103 which is fixedly attached a predetermined distance from one end of a hub 105. The fastener 103 works to maintain the cannula at a specific position on the neck but also enables the hub 105 to remain outside the trachea for attachment of the oxygen source. The fastener 103 further includes a plurality of flanges 107 that include an aperture 109 therein. Each of the respective apertures 109 is used with a tracheostomy tie (not shown) which is essentially a strap that is fastened around the patient's neck and tied to each aperture 109. The tracheostomy ties are typically secured to the flange 107 and to each other using a hook-and-loop fastener material such as VELCRO or the like. The tracheostomy tie typically is a strap that is manufactured of a cotton twill tape-like material which securely holds the tracheostomy tube 100 around the patient's neck. Additionally, an obturator 111 can be used with the tracheostomy tube 100 to stiffen the tube for smooth and easy insertion into the patient's trachea.

U.S. Patent Publication US 2002/0139372 A1 to Shikani discloses another tracheostomy tube with an adjustable quick release. The quick release is a buckle-type fastener with teeth that frictionally engage with a tube tie that passes through it. The teeth grip the tube tie until such time as the tie is moved, enabling the teeth to be released. As will be recognized by those skilled in the art, the apparatus as disclosed by Shikani is used primarily as a rapid release type of device. It is not intended to be used in an urgent or emergency situation where the tracheostomy tube is held in position and secured to the patient by a sole caregiver without the aid of other persons. Moreover, the buckle fastener mechanically is difficult to use and cannot be finely adjusted to provide a desired tube tie diameter.

Thus, the need exists for an improved tube tie that includes an adjustment mechanism that is easily adjustable by a sole caregiver without the need for tying, snapping or buckling, thereby permitting the caregiver to easily replace a tracheostomy tube in an emergency situation.

### SUMMARY OF THE INVENTION

Briefly, according to the invention, there is provided a tracheostomy tube with an improved tie and method whereby a sole caregiver can safely replace a tracheostomy tube in an emergency situation. The invention includes securing the tracheostomy tube using a tube tie that uses no hook-and-loop materials, snaps or buckles, but instead uses a barrel fastener that is slidably adjusted along a length of the tube tie, allowing the tube tie to be set to a fixed diameter. This enables the caregiver to easily replace the tracheostomy tube using only two hands, allowing the patient to remain comfortable with less stress and discomfort. In an alternative embodiment of the invention, the fastener is integrally formed within the tracheostomy tube allowing the tracheostomy tube tie to be easily held to a fixed position.

### BRIEF DESCRIPTION OF THE DRAWINGS

The features of the present invention, which are believed to be novel, are set forth with particularity in the appended claims. The invention, together with further objects and advantages thereof, may best be understood by reference to the following description, taken in conjunction with the accompanying drawings, in the several figures of which like reference numerals identify like elements, and in which:

FIG. 1 illustrates a perspective view of a tracheostomy tube as known in the prior art.

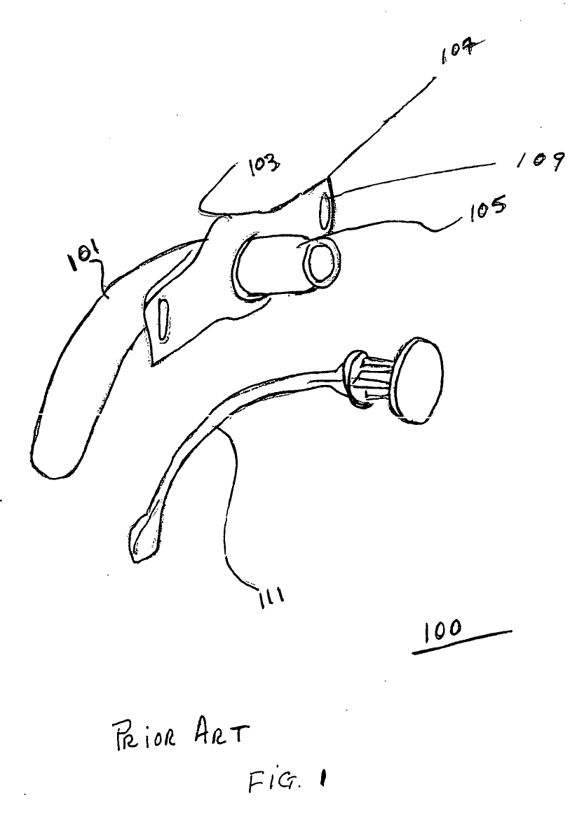

FIG. 2 illustrates inserting the tracheostomy tube into the patient's trachea.

FIG. 3 illustrates holding the tracheostomy tube on the patient's neck.

FIG. 4 illustrates positioning the tracheostomy tube tie about the patient's neck with one end inserted through a tracheostomy tube flange.

FIG. 5 illustrates sliding a barrel fastener along the tracheostomy tube tie in order to set the diameter of the tube tie in accordance with the preferred embodiment of the invention.

FIG. 6 illustrates securely holding the tracheostomy tube in position using the barrel connector as shown in FIG. 5.

FIG. 7 is a perspective view of the tracheostomy tube with improved tie according to the invention.

FIG. 8 illustrates an alternative embodiment of the invention with fastener integrally formed with tracheostomy tube.

### DETAILED DESCRIPTION OF THE PREFERRED EMBODIMENT

While the specification concludes with claims defining the features of the invention that are regarded as novel, it is believed that the invention will be better understood from a consideration of the following description in conjunction with the drawing figures, in which like reference numerals are carried forward.

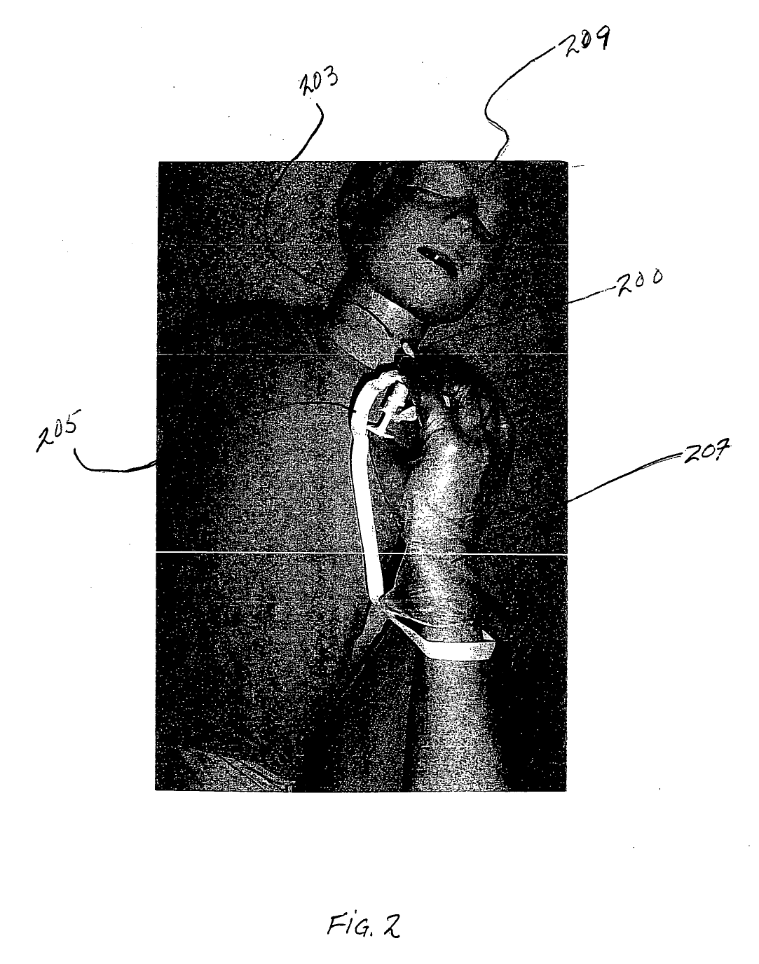

Referring now to FIGS. 2-6, the improved tracheostomy tube tie and method of holding same includes an apparatus and method for changing a tracheostomy tube that can be used by a single caregiver.

FIG. 2 illustrates inserting a tracheostomy tube 200 into the stoma 203 of the patient. The tracheostomy tube tie 205 is positioned over the caregiver's wrist so that it may easily be lifted by the caregiver's other hand (not shown) over the patient's head 209.

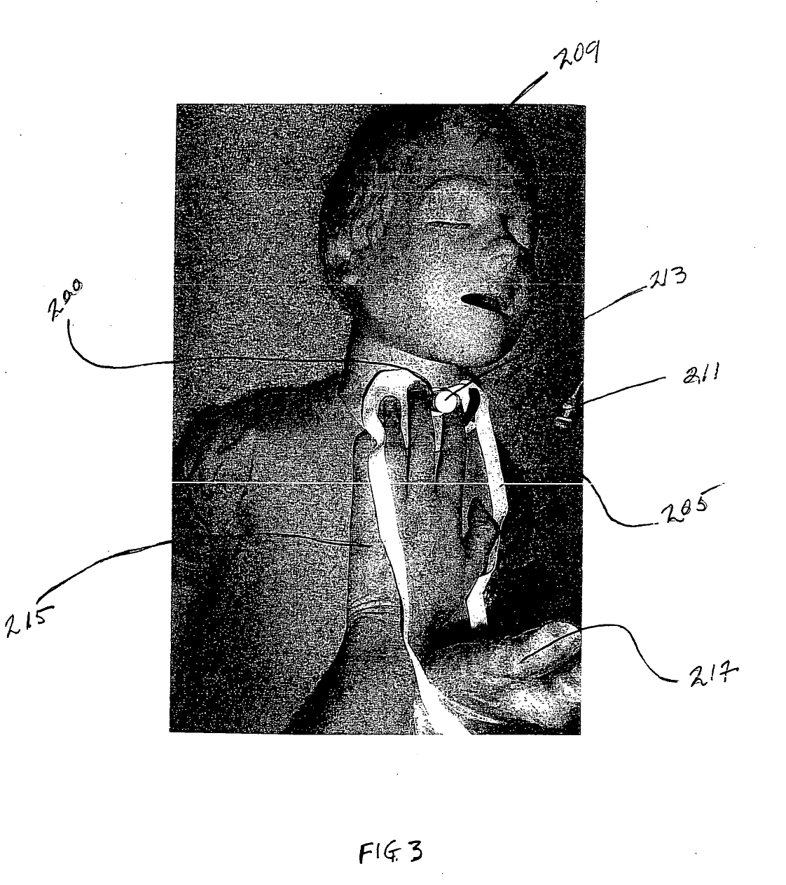

FIG. 3 illustrates the tracheostomy tube 200 positioned within the patient's trachea where the obturator 211 has been removed from the tracheostomy hub 213. In this illustration, the caregiver's left hand is used to hold the tracheostomy hub 213 in a fixed position to the patient's trachea while the caregiver's right hand 217 is used to lift the tracheostomy tie 205 so as to position the tracheostomy tie 205 around the patient's head 209.

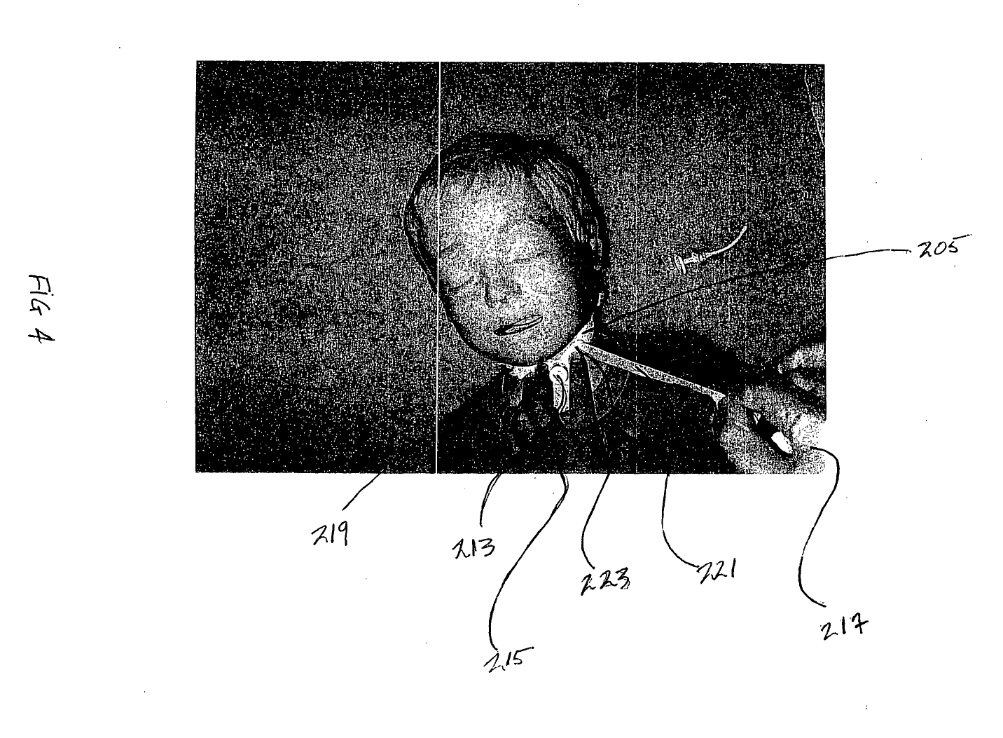

FIG. 4 illustrates the tracheostomy tube tie 205 positioned about the patient's neck 219. In this example, one end 221 of the tracheostomy tube tie 205 was threaded through the flange 223 before positioning over the patient's head. Alternatively, those skilled in the art will recognize that the one end 221 of the tracheostomy tube tie 205 may be positioned through the flange 223 after the tube tie is moved over the patient's head. In order hold the tracheostomy tube in a fixed position using the tube tie, the one end 221 of the tube tie is pulled lightly taut while the tracheostomy tube is held in position with the caregiver's opposite hand. In the example shown in FIG. 4, the caregiver's left hand 215 is used to hold the tracheostomy hub 213 in position while the caregiver's right hand 217 is used to provide tension on the one end 221 of the tracheostomy tube tie in order to adjust the tube tie to a fixed diameter.

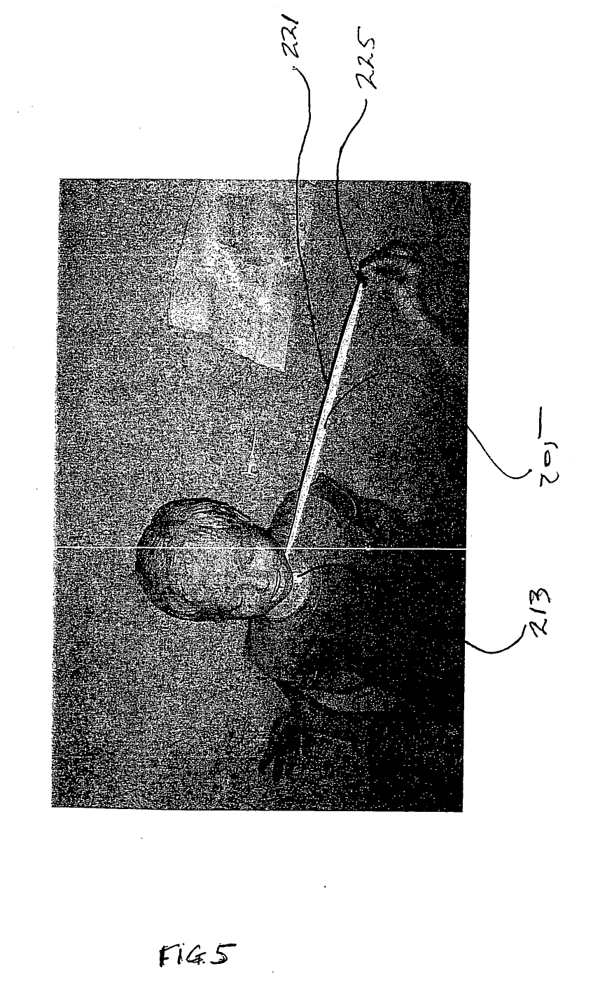

FIG. 5 illustrates slidably adjusting a barrel fastener 225 also called a “cord lock” fastener along the length of the one end of the tracheostomy tube tie 205. The barrel fastener 225 allows the tube tie 205 to pass through its body where a spring or other resistant mechanism acts to provide frictional engagement with the tube tie 205. This allows the barrel fastener 225 to be securely held in position with the tube tie 205. The barrel fastener 225 is adjusted by disengaging the spring, allowing the body of the barrel fastener 225 to move freely along the length of tube tie 205. The barrel fastener 225 is moved along the length of the tracheostomy tube tie 205 so as to adjust the diameter of the tracheostomy tube tie 205 to a desired length. Preferably, the diameter of the tracheostomy tube tie 205 is adjusted so that the tracheostomy tube hub 212 is securely held in position without providing undue tension or irritation which may bind or choke the patient. Typically, the correct tension on the tube tie 205 will allow a diameter in which at least one finger of the caregiver is able to be fit under the tube tie 205. After the tracheostomy tube tie 205 is adjusted to the proper position, an end of the tracheostomy tube tie 205 may be reduced in size by cutting off an excess in order to facilitate use of the barrel fastener 225.

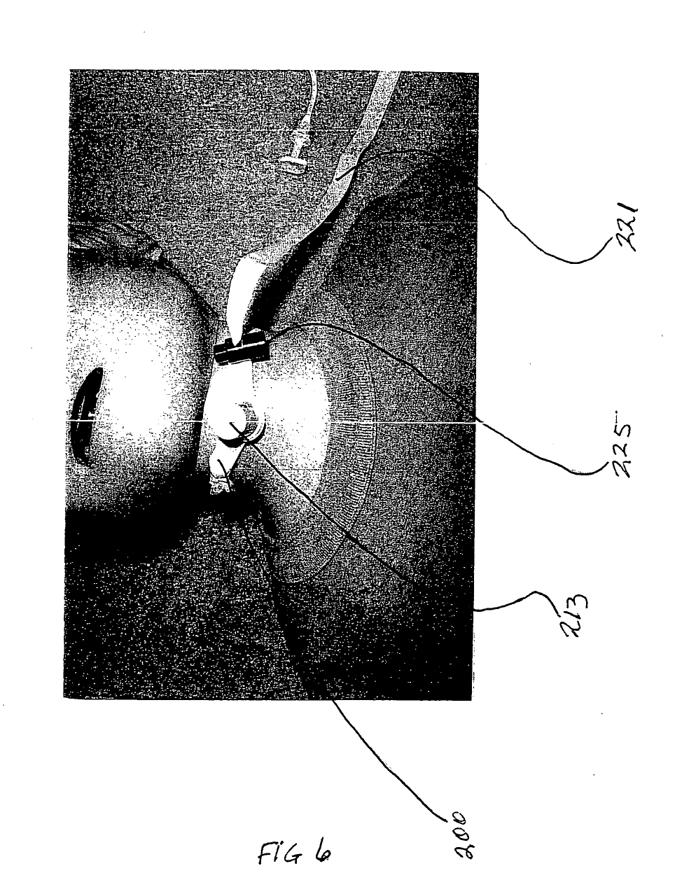

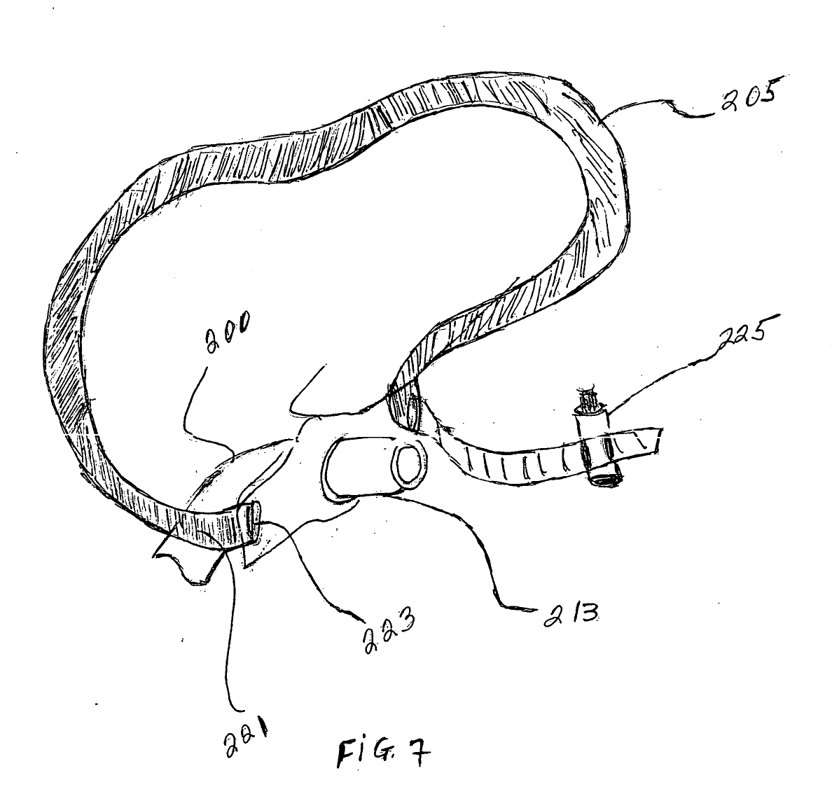

FIG. 6 illustrates the barrel fastener 225 positioned so as to hold the tracheostomy tube tie 200 in a fixed position so that it remains immovable from the patient's stoma. FIG. 7 is a perspective view of the tracheostomy tube with improved tie according to the invention. As noted in FIGS. 2-6, this illustration shows the barrel fastener 225 slidably movable along one end of the tube tie 205 which acts to hold the tube tie 205 at a fixed diameter about a patient's neck. As will be recognized by those skilled in the art, the invention provides an apparatus and method whereby a sole caregiver can change or replace a tracheostomy tube without others being present to assist. This gives great comfort when the caregiver may be alone caring for the patient and an emergency situation develops, such as a clogged tracheostomy tube or the like. The use of the barrel fastener 225 enables the tracheostomy tube tie to be easily held in position without the use of snaps, buckles, or other types of unreliable fastening materials or hardware.

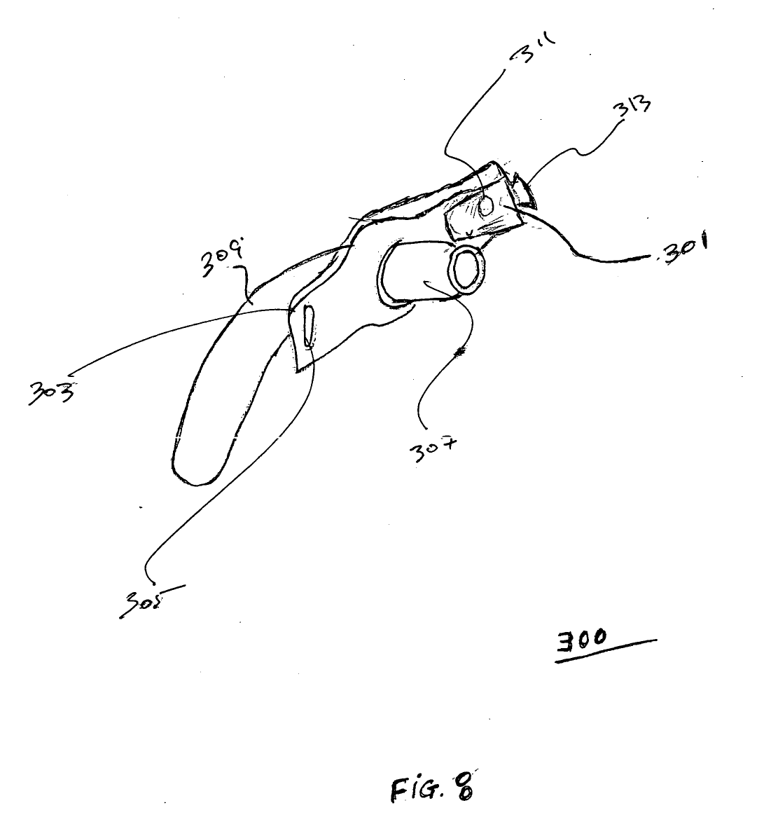

FIG. 8 illustrates an alternative embodiment of the invention where an adapted tracheostomy tube 300 includes an integrated fastener mechanism 301 that is built within flange 303. On one side of the flange 303, an aperture 305 is used for securing a tracheostomy tube tie (not shown) while the opposite side of the flange 303 includes the fastener mechanism 301. The adapted tracheostomy tube 300 further includes a hub 307 for attachment to a ventilator, oxygen source or the like. As noted herein, the cannula 309 is used for insertion into the patient's trachea. As will be recognized by those skilled in the art, the fastener mechanism 301 might be a barrel fastener or any type of mechanical fastener that frictionally engages a tracheostomy tie as it passes through fastening aperture 311. When used with a patient, the tracheostomy tube tie might be fastened to the flange 303 through aperture 305 where it then passes around the patient's neck and is positioned through the fastening aperture 311. A release button 313 is used to allow the tracheostomy tie to move freely through the fastening aperture 311 allowing the desired diameter of the tracheostomy tie to be easily selected without choking the patient. When the correct diameter is achieved, the release button 313 is released where and internal resilient mechanism or spring moved the body of the release button to frictionally engage the tracheostomy tie. This allows the tracheostomy tie to be held firmly in position so as to maintain a fixed position of the cannula 309 within the patient's trachea.

Thus, the invention provides a tracheostomy tube tie apparatus and method that includes a tracheostomy tube tie for attachment to a tracheostomy tube about a patient's neck. A barrel fastener is then attached to the tracheostomy tube tie for slidably adjusting the diameter of the tracheostomy tube tie holding it in a fixed position. The barrel fastener frictionally engages with the tracheostomy tube tie preventing it from expanding from a fixed position. In an alternative embodiment, the barrel fastener can be integrally mounted or formed within a flange of the tracheostomy tube.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as defined by the appended claims.

1. A tracheostomy tube capable of replacement by a single caregiver comprising:
a hub;
a plurality of flanges for fastening a tube tie to the hub;
a tracheostomy tube tie for securing the tracheostomy tube in a fixed position; and
wherein the tracheostomy tube tie includes a barrel fastener having an internal release mechanism integrated within at least one of the plurality of flanges for slidably holding the tracheostomy tube tie at a fixed diameter.

2. A tracheostomy tube capable of replacement by a single caregiver as in claim 1, further wherein the barrel fastener maintains a fixed diameter by preventing the tube tie from retracting back through at least one of the plurality of flanges.

3. A tracheostomy tube capable of replacement by a single caregiver as in claim 1, wherein the barrel fastener is integrally mounted within at least one of the plurality of flanges.

4. An emergency tracheostomy tube fastening system comprising:
a tracheostomy tube having a plurality of flanges;
a tracheostomy tube tie for fastening to at least one of the plurality of flanges and holding the tracheostomy tube in a substantially fixed position; and
a cylindrical cord lock fastener and release mechanism integral within at least one of the plurality of flanges for adjusting the tracheostomy tube tie to a fixed diameter.

5. An emergency tracheostomy tube fastening system as in claim 4,
wherein the cord lock fastener maintains a fixed diameter by preventing the tracheostomy tube tie from extending back though at least one of the plurality of flanges.

6. An emergency tracheostomy tube fastening system as in claim 4,
wherein the cord lock fastener is integrally mounted within at least one of the plurality of flanges.

7. A tracheostomy tube tie apparatus comprising:
a tracheostomy tube tie for attachment to a tracheostomy tube about a patient's neck; and
a cylindrical barrel fastener and release mechanism integral with a flange portion of the tracheostomy tube and attached to the tracheostomy tube tie for slidably adjusting the diameter of the tracheostomy tube tie to hold the tracheostomy tube in a fixed position.

8. A tracheostomy tube tie apparatus as in claim 7, wherein the barrel fastener frictionally engages with the tracheostomy tube tie preventing it from expanding from a the fixed position.

9. A tracheostomy tube tie apparatus as in claim 7, wherein the barrel fastener is integrally mounted within a flange of the tracheostomy tube.

10. A method allowing one caregiver to replace a tracheostomy tube without the aid of others comprising the steps of:
positioning the tracheostomy tube with a patient's trachea;
guiding a tracheostomy tube tie around the patient's neck;
attaching one end of the tracheostomy tube tie through at least one connecting portion of the tracheostomy tube; and
slidably adjusting a cord lock fastener along a section of the tracheostomy tube tie for setting the diameter of the tracheostomy tube tie to a desired length.

11. A method allowing one caregiver to replace a tracheostomy tube as in claim 10, further comprising the step of:
reducing the size on one end of the tracheostomy tube tie to a selected length in order to remove excess.

12. A method allowing one caregiver to replace a tracheostomy tube as in claim 10, wherein the cord lock fastener is integrally mounted within the at least one connection portion of the tracheostomy tube.
